(19) United States
(12) Patent Application Publication
(10) Pub. No.: US 2014/0243276 A1
(43) Pub. Date: Aug. 28, 2014

(54) SMAC MIMETIC (BIRINAPANT) FOR USE IN THE TREATMENT OF PROLIFERATIVE DISEASES (CANCER)

(71) Applicants: David Weng, Malvern, PA (US); Srinivas Chunduru, West Chester, PA (US); Martin Graham, Pottstown, PA (US)

(72) Inventors: David Weng, Malvern, PA (US); Srinivas Chunduru, West Chester, PA (US); Martin Graham, Pottstown, PA (US)

(73) Assignee: TETRALOGIC PHARMACEUTICALS CORPORATION, Malvern, PA (US)

(21) Appl. No.: 14/348,074

(22) PCT Filed: Sep. 27, 2012
(86) PCT No.: PCT/US2012/057559

§ 371 Date: Mar. 28, 2014

Related U.S. Application Data
(60) Provisional application No. 61/541,531, filed on Sep. 30, 2011.
(60) Provisional application No. 61/554,829, filed on Nov. 2, 2011.
(60) Provisional application No. 61/559,058, filed on Nov. 12, 2011.
(60) Provisional application No. 61/656,026, filed on Jun. 6, 2012.

(51) Int. Cl. A61K 38/05; A61K 45/06

(52) U.S. Cl. CPC A61K 38/05; A61K 45/06
USPC 514/19.4; 514/19.3; 514/19.5; 514/19.6; 514/19.8; 514/21.91

(57) ABSTRACT

A method of using a Smac mimetic and pharmaceutical compositions thereof.

### CROSS REFERENCE TO RELATED APPLICATIONS

This applications claims priority to U.S. Provisional Application No. 61/541,531, filed Sep. 30, 2011; U.S. Provisional Application No. 61/554,829, filed Nov. 2, 2011; U.S. Provisional Application No. 61/559,058, filed Nov. 12, 2011 and U.S. Provisional Application No. 61/656,026, filed Jun. 6, 2012, all of which are incorporated herein in their entirety by reference.

### FIELD OF THE INVENTION

This invention is in the field of Smac mimetics and compositions and uses thereof to treat proliferative disorders including cancers.

### BACKGROUND OF THE INVENTION

Inhibitors of Apoptosis Proteins (IAPs) are naturally occurring intra-cellular proteins that suppress caspase-dependent apoptosis. Smac, also known as DIABLO, is another intracellular protein that functions to antagonize, i.e., inhibit the activity of IAPs. In normal healthy cells, Smac and IAPs function together to maintain the viability of healthy cells. However, in certain disease states, e.g., cancers and other proliferative disorders, IAPs are not adequately antagonized and therefore prevent apoptosis and cause or exacerbate abnormal proliferation and survival.

Smac mimetics, also known as IAP antagonists, are synthetic small molecules that mimic the structure and IAP antagonist activity of the four N-terminal amino acids of Smac. (Smac mimetics are sometimes referred to as IAP antagonists.) When administered to animals suffering proliferative disorders, the Smac mimetics antagonize IAPs, causing an increase in apoptosis among abnormally proliferating cells.

Examples of Smac peptidomimetics are those disclosed in, without limitation, U.S. Pat. No. 7,517,906; U.S. Pat. No. 7,419,975; U.S. Pat. No. 7,589,118; U.S. Pat. No. 7,932,382; U.S. Pat. No. 7,345,081; U.S. Pat. No. 7,244,851; U.S. Pat. No. 7,674,787; U.S. Pat. No. 7,772,177; U.S. Pat. No. 7,989,441; US20100324083; US20100056467; US20090069294; US20110065726; US20110206690; WO2011098904.

### SUMMARY OF THE INVENTION

This invention, in one aspect, is a method of treating a patient suffering a proliferative disorder that comprises administering a selected dose, including a high dose relative to previously understood doses, of N-{1S-[2R-(6,6′-Difluoro-3′-{4S-hydroxy-1-[2S-(2S-methylamino-propionylamino)-butyryl]-pyrrolidin-2R-ylmethyl}-1H,1′H-[2,2′]biindolyl-3-ylmethyl)-4 S-hydroxy-pyrrolidine-1-carbonyl]-propyl}-2S-methylamino-propionamide and pharmaceutically acceptable salts thereof, as well as various forms of such compound and salts thereof as further described herein below.

This compound is disclosed in US20110003877, the entire disclosure of which is hereby incorporated by reference as though fully set forth herein, and the compound has the following structure:

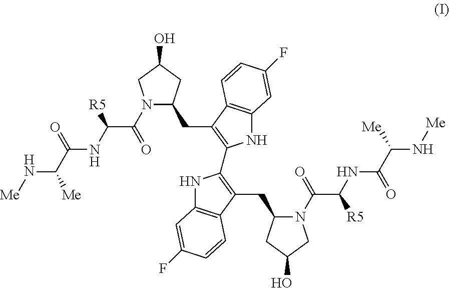

wherein R5 is —CH2CH3 and Me is methyl. This compound is also referred to herein as Compound 15. It is also known as birinapant.

The invention, in related aspects, comprises a pharmaceutical composition in a dosage unit for intravenous infusion comprising such compound in a dose as hereinafter described and a method of treating a proliferative disorder in a human or non-human mammalian subject in need thereof that comprises internally administering to the subject an effective amount of said compound or a pharmaceutically acceptable salt thereof wherein the effective amount is a dose as defined more fully hereinafter.

In additional illustrative embodiments, the invention comprises a method of potentiating apoptosis of abnormally proliferating cells in a human or non-human mammalian subject that comprises internally administering, e.g., by intravenous infusion, a hereinafter defined dose of Compound 15.

In additional illustrative embodiments, the invention comprises any one or more of the above methods that further comprises administering a second cancer-related therapy, such as, e.g., radiation, chemotherapy, immunotherapy, photodynamic therapy, and combinations thereof.

In a further illustrative embodiment, the invention comprises a method of treating an autoimmune disease, in which the condition is caused or exacerbated by abnormal regulation of apoptosis, in a mammal in need thereof, including, for example, systemic lupus erythematosus, psoriasis, and immune thrombocytopenic purpura that comprises internally administering to the animal a hereinafter defined dose of Compound 15 or a pharmaceutically acceptable salt thereof.

### DETAILED DESCRIPTION OF THE INVENTION

The compound administered in accordance with the present invention is a Smac mimetic that can be used in the treatment of proliferative disorders, e.g.: various benign tumors or malignant tumors (cancer), benign proliferative diseases (e.g., psoriasis, benign prostatic hypertrophy, and restenosis), or autoimmune diseases (e.g., autoimmune proliferative glomerulonephritis, lymphoproliferative autoimmune responses). Cancers which potentially can be treated with Smac mimetics, i.e., IAP antagonists, include, but are not limited to, one or more of the following: lung adenocarcinoma, pancreatic cancer, colon cancer, ovarian cancer, breast cancer, mesothelioma, peripheral neuroma, bladder cancer, glioblastoma, melanoma, adrenocortical carcinoma, AIDS-related lymphoma, anal cancer, bladder cancer, meningioma, glioma, astrocytoma, breast cancer, cervical cancer, chronic myeloproliferative disorders (e.g., polycythemia rubra vera, chronic myelogenous leukemia), chronic lymphocytic leukemia, colon cancer, endocrine cancers, endometrial cancer, ependymoma, esophageal cancer, Ewing's sarcoma, extracranial germ cell tumors, extragonadal germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, intraocular melanoma, islet cell carcinoma, Kaposi sarcoma, laryngeal cancer, leukemia, acute lymphoblastic leukemia, acute myeloid leukemia, lip cancer, oral cavity cancer, liver cancer, male breast cancer, malignant mesothelioma, medulloblastoma, melanoma, Merkel cell carcinoma, metastatic squamous neck cancer, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, oropharyngeal cancer, bone cancers, including osteosarcoma and malignant fibrous histiocytoma of bone, ovarian epithelial cancer, ovarian germ cell tumors, ovarian low malignant potential tumors, pancreatic cancer, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, skin cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

Some embodiments of the invention include inducing apoptosis of cells, particularly pathologically proliferating cells. The methods can be carried out in vitro or in vivo.

The methods of the invention can include administration of Compound 15 alone, administration of a combination of IAP antagonists, or administration of Compound 15, with or without one or more additional IAP antagonists, and one or more additional chemotherapeutic agents. Administration of multiple agents can be simultaneous or sequential. Useful chemotherapeutic agents include, but are not limited to, alkylating agents (e.g., cyclophosphamide, mechlorethamine, chlorambucil, melphalan), anthracyclines (e.g., daunorubicin, doxorubicin, epirubicin, idarubicin, mitoxantrone, valrubicin), cytoskeletal disruptors (e.g., paclitaxel, docetaxel), epothilones (e.g., epothilone A, epothilone B, epothilone D), inhibitors of topoisomerase I and II (e.g., irinotecan, topotecan, etoposide, teniposide, tafluposide), nucleotide analogs precursor analogs (e.g., azacytidine, azathioprine, capecitabine, cytarabine, doxifluridine, fluorouracil, gemcitabine, mercaptopurine, methotrexate, tioguanine), peptide antibiotics (e.g., bleomycin), platinum-based agents (e.g., carboplatin, cisplatin, oxaliplatin), retinoids (e.g., all-trans retinoic acid), and vinca alkaloids and derivatives (e.g., vinblastine, vincristine, vindesine, vinorelbine). In some embodiments, chemotherapeutic agents include fludarabine, doxorubicin, paclitaxel, docetaxel, camptothecin, etoposide, topotecan, irinotecan, cisplatin, carboplatin, oxaliplatin, amsacrine, mitoxantrone, 5-fluoro-uracil, or gemcitabine.

In some embodiments of the invention, pharmaceutical compositions comprising Compound 15, alone or in combination with one or more other active pharmaceutical ingredients, are administered to a human or veterinary subject. The pharmaceutical compositions typically comprise at least one pharmaceutically acceptable excipient, e.g., a carrier or diluent, and can be administered in the conventional manner by routes including systemic, topical, or oral routes. Administration is normally by intravenous injection, either as a bolus or infusion, but other routes of administration are not precluded including, e.g., subcutaneous, intramuscular, intraperitoneal, intrapleural, intrathecal, intraorbital, or intraarterial injection. An intravenous formulation can contain, e.g., from 1 mg/mL up to and including 5 mg/mL of Compound 15 in sterile 0.05M citrate buffered saline, pH 5. For intravenous infusion, Compound 15, e.g., 1 mg/mL or 5 mg/mL in 0.05M citrate buffered saline, can be added to sterile saline in an infusion bag in an amount calculated to deliver the desired dose.

Typically, Compound 15 will be administered by intravenous infusion, including, e.g., by infusion over an infusion period of about 1 to about 120 minutes, or 1 to about 60 minutes, e.g., about 30 minutes.

The pharmaceutical composition of the invention is a composition in which the active pharmaceutical ingredient, i.e., Compound 15, is pure enough, and the composition is otherwise suitable, for internal administration to a human or other mammal. It can be prepared in unit dose form, i.e., a form suitable for single administration to a subject such as by infusion. So, e.g., a pharmaceutical composition in intravenous unit dose form may comprise a vial or pre-filled syringe, or an infusion bag or device, each comprising a sufficient amount of Compound 15 to supply the desired dose (or a convenient fraction of such dose), as described hereinafter, such that the contents of one vial or syringe (or a small number of multiple vials, depending upon the fraction of dose in each) are administered at a time.

Administration can be repeated up to about 4 times per day over a period of time, if necessary to achieve a cumulative effective dose, e.g., a cumulative dose effective to produce tumor stasis or regression. A dosing regimen can be, e.g., daily, twice-weekly, or three times weekly (i.e., thrice weekly) intravenous injections, or, e.g., once weekly injections in cycles of three weeks on and one week off, or continuously, for as long as the treatment is effective, e.g., until disease progresses or the drug is not tolerated. The effective dose administered in each injection is an amount that is effective and tolerated.

An effective dose is one that over the course of therapy, which may be, e.g., 1 or more weeks, e.g., multiple courses of 3 weeks on/1 week off, results in treatment of the proliferative disorder, i.e., a decrease in the rate of disease progression, termination of disease progression, or regression or remission.

It has been found as an aspect of this invention that Compound 15 is unexpectedly well tolerated. In some embodiments of the invention, Compound 15 can therefore, in general, be administered in doses that are higher than previously understood (see, e.g., US20110003877). In some embodiments of the invention, Compound 15 can, in general, be administered in doses that are generally higher than other synthetic small molecules that mimic the structure and IAP antagonist activity of the four N-terminal amino acids of Smac (i.e., other Smac mimetics). Other Smac mimetics have lower maximum tolerated doses (MTD) and have not shown meaningful clinical efficacy below such MTDs.

Doses employed in the practice of this invention can be effective in potentiating apoptosis of abnormally proliferating cells in a patient suffering a proliferative disorder or certain other disorders, e.g., certain autoimmune disorders. For example, Compound 15 can be administered intravenously, e.g., by infusion, at a dose of 1 to 80 mg/m² of patient body surface area (BSA) per day of treatment, e.g., 2 to 80, 2 to 65, 5 to 65, 10 to 65, 20 to 65, 30 to 65, 30 or >30 to 80, 30 or >30 to 65, 30 or >30 to 60, 30 or >30 to 55, or 30 or >30 to 50 mg/m², administered, e.g., by infusion over about 1 to about 120 minutes, e.g., about 30 minutes. The dose in most cases will be more than 5 mg/m². For example, the dose can be in the range 5 or >5 to 80, 5 or >5 to 60 mg/m². Current clinical studies employ about 5 mg/m² to about 50 mg/m², specifically, 5.6 to 47 mg/m². In two patients who received 63 mg/m², weekly/3 weeks on, /1 week off, Compound 15 was not well tolerated.

It will be understood that there are different formulae for calculating BSA. Most commonly used are the Mosteller formula (Mosteller R D. “Simplified calculation of body-surface area”. N Engl J Med 317:1098 (1987)) and the Dubois & Dubois formula (Du Bois & Du Bois, Arch Intern Med 17:863 (1916)). Doses recited herein are meant to apply to BSA calculated as per any such accepted methodologies notwithstanding that such different methodologies may result in slightly different BSA calculations, e.g., depending upon the number of decimal places used. It is generally sufficient to round off BSA calculations to 1 decimal place with allowance for a reasonable margin of error, e.g., 1.6 m² (+/−0.1) or 1.9 m² (+/−0.1). For purposes of this invention, BSA can also be estimated, e.g., using relevant population averages.

Doses recited herein as mg/m² BSA can, of course, be converted to mg/kg body weight. So, for example, assuming a given patient has a BSA of 1.6 m² and a body weight of 77 kg, a dose of 40 mg/m² is equal to a dose of 64 mg, i.e., about 0.8 mg/kg. By way of further example, using an average adult BSA of 1.7 m² and an average adult body weight of 70 kg, a dose of 40 mg/m² is equal to a dose of 68 mg, i.e., also about 0.8 mg/kg. Similarly, a dose range of >30 to 60 mg/m² equates to a dose range of >0.7 mg/kg to approximately 1.5 mg/kg, in such person of average BSA and weight.

It has also been discovered that Compound 15 has a long half-life in the patient and therefore can be administered less often than once per day. In general, Compound 15 can be administered once, twice or three times per week for one to four weeks (or longer). In some situations a treatment interval may be followed by a rest interval. A suitable rest interval includes but is not limited to one week. Such treatment cycle of one, two, three or four weeks “on” and one week “off” can be continued for as long as Compound 15 shows effectiveness and is tolerated. It should be understood that the “on” weeks are consecutive weeks, i.e., two consecutive weeks on drug, three consecutive weeks on drug, and four consecutive weeks (or more) on drug.

An illustrative dosing regimen for Compound 15 is one ˜30 minute infusion/week for one to four weeks, e.g., once a week for 2 or 3 consecutive weeks, followed by a week off. Specific illustrative dosing regimens include, without limitation, one administration by, e.g., intravenous infusion, of drug per week, in accordance with one of the following treatment cycles:
1) two weeks on/one week off, e.g., in combination with chemotherapies;
2) one week on/one week off, e.g., in patients with AML;
3) two weeks on/one week off, e.g., in patients with AML;
4) three weeks on/one week off, e.g., in patients with AML;
5) continuously (i.e., without a rest interval).

An illustrative dosing regimen for Compound 15 is one 30 minute infusion/week for 2 to 4 weeks, e.g., once a week for 2 or 3 consecutive weeks, followed by a week off. Such treatment cycle of two, three or four weeks on and one week off can be continued for as long as Compound 15 shows effectiveness and is tolerated.

In an alternative dosing regimen, Compound 15 is administered weekly, twice weekly, or three times per week, without a rest interval, i.e., continuously, for as long as Compound 15 shows effectiveness and is tolerated.

It is noteworthy and a priori unpredictable that a dose of >30 mg/m², e.g., >30 to 65, >30 to 60 or >30 to 50 mg/m², can be tolerated and effective when administered by intravenous infusion during a period of about 30 minutes once per week for three or four weeks on and one week off or continuously.

Typically, higher doses will be employed when Compound 15 is used in monotherapy, i.e., single agent therapy, then in combination therapy. Such monotherapy dose can be, e.g., about 40 to about 55 mg/m², or about 45 to about 50 mg/m², weekly for three weeks on/one week off or weekly continuously. An illustrative dosing regimen for Compound 15 in single agent therapy is 45 to 50 mg/m², e.g., 47 mg/m², weekly for three weeks on/one week off or weekly continuously.

When Compound 15 is used in combination therapy, the dose can be, e.g., about 5 to about 50 mg/m², or about 5 to about 40 mg/m², weekly for three weeks on/one week off or weekly continuously. An illustrative dosing regimen for Compound 15 in combination therapy is about 5 to about 35 mg/m², weekly for three weeks on/one week off or weekly continuously.

In patients in whom Compound 15 is less well tolerated, lower doses can be administered more frequently. For example, in AML patients, Compound 15 can be administered in single agent therapy at about 15 to about 20 mg/m², e.g., 17 mg/m², twice/week (e.g., Mondays and Thursdays, Tuesdays and Fridays, etc.) or 17 mg mg/m², thrice/week (e.g., Mondays, Wednesdays, Fridays). three weeks on/one week off or continuously.

The phrase “pharmaceutical composition” refers to a composition suitable for administration in a medical use, i.e., internal administration to a patient. Compositions suitable for infusion in accordance with the method of this invention conveniently comprise a sterile aqueous preparation of Compound 15, which is preferably isotonic with the blood of the recipient. This aqueous preparation may be formulated according to known methods using suitable carriers or diluents which may include a buffer. Thus, in one illustrative aspect, this invention comprises a pharmaceutical dosage unit comprising Compound 15 and one or more pharmaceutically acceptable excipients in an aqueous solvent for use in intravenous or subcutaneous administration for the treatment of a cancer or an autoimmune disorder.

When practicing the conjoint or combination therapy described in more detail below, the administration of Compound 15 can occur simultaneous with, subsequent to, or prior to the combination therapy, such as chemotherapy or radiation, so long as the chemotherapeutic agent or radiation sensitizes the system to the method and compositions of the present invention.

The present invention also is directed to the use of Compound 15 as a chemopotentiating agent with other treatment approaches. The term “chemopotentiating agent” refers to an agent that acts to increase the sensitivity of an organism, tissue, or cell to a chemical compound, or treatment namely “chemotherapeutic agents” or “chemo drugs” or to radiation treatment. Thus, the methods and compositions of the present invention can be used for inhibiting tumor growth in vivo by administering them in combination with a biologic or chemotherapeutic agent or by using them in combination with radiation. In these applications, the administration of Compound 15 in accordance with the present invention may occur prior to, and with sufficient time, to cause sensitization of the site to be treated. Alternatively, Compound 15 may be used contemporaneously with radiation and/or additional anti-cancer chemical agents (infra).

Biological and chemotherapeutics/anti-neoplastic agents and radiation induce apoptosis by activating the extrinsic or intrinsic apoptotic pathways, and, since the method and compositions of the present invention relieve antagonists of apoptotic proteins (IAPs) and, thus, remove the block in apoptosis, the combination of chemotherapeutics/anti-neoplastic agents and radiation with the method and compositions of the present invention should work additively or synergistically to facilitate apoptosis.

A combination of the compound of the present invention and a biological or chemotherapeutic/anti neoplastic agent and/or radiation therapy of any type that activates the extrinsic or intrinsic pathway may provide a more effective approach to destroying tumor cells. The compound of the present invention interacts with IAP's, such as XIAP, cIAP-1, cIAP-2, ML-IAP, etc., and removes the IAP mediated block of apoptosis. Most chemotherapeutics/anti neoplastic agents and/or radiation therapy kills actively dividing cells by activating the intrinsic apoptotic pathway leading to apoptosis and cell death. Biological antitumor agents such as TRAIL (TNF-related apoptosis inducing ligand) activate extrinsic apoptotic pathways. As is described in more detail below, embodiments of the invention provide combinations of the compound of the present invention and a biological or chemotherapeutic/anti-neoplastic agent and/or radiation which provide a synergistic action against unwanted cell proliferation. This synergistic action between the compound of the present invention and a biological or chemotherapeutic/anti-neoplastic agent and/or radiation therapy can improve the efficiency of the biological or chemotherapeutic/anti-neoplastic agent and/or radiation therapies. This will allow for an increase in the effectiveness of current biological or chemotherapeutic/anti-neoplastic agents or radiation treatments allowing a higher percentage of tumors to respond to the therapy, an improved tumor response, and, potentially, a reduction in the dose of the biological or chemotherapeutic/anti-neoplastic agent needed to treat a tumor, thereby providing the use of a more tolerable dose of biological or chemotherapeutic/anti-neoplastic agent and/or radiation.

In an embodiment of the present invention, the patient is treated by administering the compound or a pharmaceutical composition of the present invention at a time the patient is subject to concurrent or antecedent radiation or chemotherapy for treatment of a neoproliferative pathology of a tumor such as, but not limited to, bladder cancer, breast cancer, prostate cancer, lung cancer, pancreatic cancer, gastric cancer, colon cancer, ovarian cancer, renal cancer, hepatoma, melanoma, lymphoma, sarcoma, and combinations thereof.

In another embodiment of the present invention, the compound or a composition of the present invention can be administered in combination with a biological or chemotherapeutic and/or for use in combination with radiotherapy, immunotherapy, and/or photodynamic therapy, promoting apoptosis and enhancing the effectiveness of the chemotherapeutic, radiotherapy, immunotherapy, and/or photodynamic therapy.

As discussed above, embodiments of the invention also include a method of treating a patient afflicted with cancer by the contemporaneous or concurrent administration of a biological or chemotherapeutic agent additional to Compound 15. Such biological or chemotherapeutic agents include but are not limited to the chemotherapeutic agents described in “Modern Pharmacology with Clinical Applications”, Sixth Edition, Craig & Stitzel, Chpt. 56, pg 639-656 (2004), herein incorporated by reference in its entirety. The chemotherapeutic agent can be, but is not limited to, alkylating agents, antimetabolites, anti-tumor antibiotics, plant-derived products such as taxanes, enzymes, hormonal agents, miscellaneous agents such as cisplatin, monoclonal antibodies, glucocorticoids, mitotic inhibitors, topoisomerase I inhibitors, topoisomerase II inhibitors, immunomodulating agents such as interferons, cellular growth factors, cytokines, and nonsteroidal anti-inflammatory compounds (NSAID), cellular growth factors and kinase inhibitors. Other suitable classifications for chemotherapeutic agents include mitotic inhibitors, and anti-estrogenic agents.

Specific examples of suitable biological and chemotherapeutic agents include, but are not limited to, carboplatin, cisplatin, carmustine (BCNU), bendamustine, 5-fluorouracil (5-FU), cytarabine (Ara-C), clofarabine, decitabine, 5-azacytidine, gemcitabine, methotrexate, daunorubicin, doxorubicin, dexamethasone, irinotecan, topotecan, etoposide, paclitaxel, docetaxel, vincristine, tamoxifen, TNF-alpha, TRAIL and other members, i.e., other than TRAIL and TNF-alpha, of the TNF superfamily of molecules, interferon (in both its alpha and beta forms), GM-CSF, IL-2, thalidomide, thalidomide derivatives such as lenalidomide, melphalan, inhibitors of kinase enzymes such as EGFR, Her-2, B-RAF, ALK, Met encompassing both small molecules and antibodies, and PARP inhibitors. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, and supportive care agents such as erythropoietin, romiplostim, eltrombopag, filgrastim and sargramostim.

Another embodiment of the present invention relates to the use of the compound or a composition of the present invention in combination with topoisomerase inhibitors to potentiate their apoptotic inducing effect. Topoisomerase inhibitors inhibit DNA replication and repair, thereby promoting apoptosis and are used as chemotherapeutic agents. Topoisomerase inhibitors promote DNA damage by inhibiting the enzymes that are required in the DNA repair process. Therefore, export of Smac from the mitochondria into the cell cytosol is provoked by the DNA damage caused by topoisomerase inhibitors. Topoisomerase inhibitors of both the Type I class (camptothecin, topotecan, SN-38 (irinotecan active metabolite) and the Type II class (etoposide) are expected to show potent synergy with compounds of the present invention. Further examples of topoisomerase inhibiting agents that may be used include, but are not limited to, irinotecan, topotecan, etoposide, amsacrine, exatecan, gimatecan, etc. Other topoisomerase inhibitors include, for example, Aclacinomycin A, camptothecin, daunorubicin, doxorubicin, ellipticine, epirubicin, and mitaxantrone.

Another embodiment of the present invention relates to the use of the compound or a composition of the present invention in combination with nonsteroidal antiinflammatory drugs (NSAIDs).

In another embodiment of the invention, the chemotherapeutic/anti-neoplastic agent for use in combination with the method and compositions of the present invention may be a platinum containing compound. In one embodiment of the invention, the platinum containing compound is cisplatin. Cisplatin can synergize with a compound of the present invention and potentiate the inhibition of an IAP, such as but not limited to XIAP, cIAP-1, c-IAP-2, ML-IAP, etc. In another embodiment a platinum containing compound is carboplatin. Carboplatin can synergize with a compound of the present invention and potentiate the inhibition of an IAP, including, but not limited to, XIAP, cIAP-1, c-IAP-2, ML-IAP, etc. In another embodiment a platinum containing compound is oxaliplatin. The oxaliplatin can synergize with a compound of the present invention and potentiate the inhibition of an IAP, including, but not limited to, XIAP, cIAP-1, c-IAP-2, ML-IAP, etc.

Platinum chemotherapy drugs belong to a general group of DNA modifying agents. DNA modifying agents may be any highly reactive chemical compound that bonds with various nucleophilic groups in nucleic acids and proteins and cause mutagenic, carcinogenic, or cytotoxic effects. DNA modifying agents work by different mechanisms, disruption of DNA function and cell death; DNA damage/the formation of cross-bridges or bonds between atoms in the DNA; and induction of mispairing of the nucleotides leading to mutations, to achieve the same end result. Three non-limiting examples of a platinum containing DNA modifying agents are cisplatin, carboplatin and oxaliplatin.

Yet another embodiment of the present invention is the therapeutic combination or the therapeutic use in combination of the compound or compositions of the present invention with TRAIL or TRAIL agonist antibodies, or other chemical or biological agents which bind to and activate the TRAIL receptor(s). Many cancer cell types are sensitive to TRAIL-induced apoptosis, while most normal cells appear to be resistant to this action of TRAIL. TRAIL-resistant cells may arise by a variety of different mechanisms including loss of the receptor, presence of decoy receptors, overexpression of cFLIP_{L} which competes for zymogen caspase-8 binding during DISC formation and inhibition of activated caspase-3 and/or caspase-9 by XIAP. In TRAIL resistance, a compound or composition of the present invention may increase tumor cell sensitivity to TRAIL leading to enhanced cell death, the clinical correlations of which are expected to be increased apoptotic activity in TRAIL resistant tumors, improved clinical response, increased response duration, and ultimately, enhanced patient survival rate.

In another embodiment of the invention, Compound 15 is administered in combination with a cytokine, e.g., TNFα IFN, IL-2, or GM-CSF.

The method and compositions of the present invention also can be used to augment radiation therapy (or radiotherapy), i.e., the medical use of ionizing radiation as part of cancer treatment to control malignant cells. Although radiotherapy is often used as part of curative therapy, it is occasionally used as a palliative treatment, where cure is not possible and the aim is for symptomatic relief. Radiotherapy is commonly used for the treatment of tumors. It may be used as the primary therapy. It is also common to combine radiotherapy with surgery and/or chemotherapy. The most common tumors treated with radiotherapy are breast cancer, prostate cancer, rectal cancer, head & neck cancers, gynecological tumors, bladder cancer and lymphoma. Radiation therapy is commonly applied just to the localized area involved with the tumor. Often the radiation fields also include the draining lymph nodes. It is possible but uncommon to give radiotherapy to the whole body, or entire skin surface. Radiation therapy is usually given daily for up to 35-38 fractions (a daily dose is a fraction). These small frequent doses allow healthy cells time to grow back, repairing damage inflicted by the radiation. Three main divisions of radiotherapy are external beam radiotherapy or teletherapy, brachytherapy or sealed source radiotherapy and unsealed source radiotherapy, which are all suitable examples of treatment protocol in the present invention. The differences relate to the position of the radiation source; external is outside the body, while sealed and unsealed source radiotherapy has radioactive material delivered internally. Brachytherapy sealed sources are usually extracted later, while unsealed sources are injected into the body.

Compound 15 is capable of forming pharmaceutically acceptable salts, including but not limited to acid addition and/or base addition salts. Such salts are included within all aspects of the invention.

The present invention can also be practiced using isotopically-enriched compounds, which are identical to Compound 15 but for the fact that one or more atoms are replaced by an atom having an atomic mass or mass number different from the atomic mass or mass number usually found in nature. Examples of isotopes that can be included in the invention include isotopes of hydrogen, carbon, nitrogen, oxygen, phosphorous, fluorine and chlorine, such as ²H, ³H, ¹³C, ¹⁴C, ¹⁵N, ¹⁶O, ¹⁷O, ³¹P, ³²P, ³⁵S, ¹⁸F, and ³⁶Cl. Substitution with heavier isotopes such as deuterium, i.e., ²H, are also included. Isotopically enriched compounds can generally be prepared by substituting a readily available isotopically labelled reagent for a non-isotopically enriched reagent. For example, incorporation of deuterium can be accomplished by substituting sodium borohydride with d4-sodium borohydride, or by replacing iodomethane with d3-iodomethane. Representative examples of specific deuterated analogs and their preparation are described in US20110003877.

Compound 15 may exist in unsolvated forms as well as solvated forms, including hydrated forms. Furthermore, Compound 15 may exist in various solid states including crystalline, semi-crystalline and amorphous (noncrystalline) forms, and in the form of clathrates, prodrugs, polymorphs, bio-hydrolyzable esters, racemic mixtures, non-racemic mixtures, or as purified stereoisomers including, but not limited to, optically pure enantiomers and diastereomers. In general, all of these and other such forms are intended to be encompassed within the scope of the term, “Compound 15”.

References to Compound 15 in this specification and in the claims, are intended to include not only the compound of formula (I), but also pharmaceutically acceptable salts of Compound 15, as well as various forms of said compound or salts thereof such as those that are described above and below.

### EXAMPLES

### Example 1

### Illustrative Synthesis of Compound 15

The following preparations and schemes are illustrative of synthesis of Compound 15, also known as TL32711 and also as birinapant. Abbreviations which are used throughout these schemes and in the application generally, are identified in the Table 1:

TABLE 1

| ABBREVIATION | MEANING | ABBREVIATION | MEANING |
|---|---|---|---|
| ACN | Acetonitrile | NMP | N-methylpyrrolidinone |
| Ac₂O | Acetic anhydride | PhCOCl | Benzoyl chloride |
| Cbz and Z | Benzyloxycarbonyl | DIAD | diisopropyl azo dicarboxylate |
| Boc | tert-butyloxycarbonyl | DIBAL | Diisobutylaluminium hydride |
| and/or | | | |
| boc | | | |
| THF | Tetrahydrofuran | DMAP | 4-dimethylamino pyridine |
| DCM | Dichloromethane | DMF | Dimethylformamide |
| DDQ | 2,3-dichloro-5,6-dicyano-1,4- | DMSO | dimethyl sulfoxide |
| | benzoquinone | | |
| mCPBA | 3-chloroperbenzoic acid | TFA | trifluoroacetic acid |
| Cbz-Cl | Benzyloxycarbonyl chloride | TFAA | trifluoroactic anhydride |
| Hex | Hexanes | HOAc or | acetic acid |
| | | AcOH | |
| HPLC | high performance liquid | DIPEA | Diisopropylethylamine |
| | chromatography | | |
| TLC | thin layer chromatography | NMM | N-methylmorpholine |
| EtOAc | ethyl acetate | NCS | N-chlorosuccinimide |
| Ph | Phenyl | TEA (Et₃N) | Triethylamine |
| HATU | 2-(7-Aza-1H-benzotriazole-1-yl)- | MsCl | Methane-sulfonylchloride |
| | 1,1,3,3 -tetramethyluronium | | |
| | hexafluorophosphate | | |
| Me | Methyl* | Et | Ethyl |
| iPr | Iso-propyl | tBu or tert-Bu | tert-butyl |
| cPr | Cyclopropyl | cHex | Cyclohexyl |
| (2R-EtOMe) and/or R-MeCHOMe | 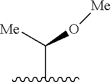 [SMILES: *[C@@H](C)OC] | (2R-EtOH) and/or R-MeCHOH | 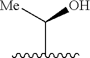 [SMILES: *[C@@H](C)O] |
| TBAF | tetrabutyl ammonium fluoride | MsCl | Methanesulfonyl chloride |
| TBDMSCl | tert-butyl-dimethyl-silyl chloride | OTBS | tert-butyl-dimethyl-silanyloxy |
| Ph₃P | Triphenylphosphine | Ac | Acetyl ( ) |
| n-Bu | Normal butyl | DMA | Dimethylamine |
| TBA-Cl | Tetra-n-butyl ammonium chloride | DMS | Dimethylsulfide |
| NP-HPLC | Normal phase-high performance | Meldrum's Acid | 2,2-dimethyl-1,3-dioxane-4,6- |
| | liquid chromatography | | dione |
| MeNO₂ | Nitromethane | MeOH | Methanol |
| EtOH | Ethanol | NaOAc | Sodium acetate |
| DCE, or EDC | Dichloroethane, Ethylenedichloride | ClCO₂Me | Ethyl chloroformate |
| Boc-N(Me)Ala-OH | 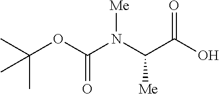 | Boc-Abu-OH | 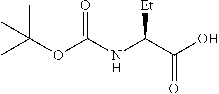 |
| NaOMe | Sodium methoxide | PSI | Pounds per Square Inch |
| | | | (Gauge) |
| h | hour | > | Greater than |

### Example 1

### Synthesis

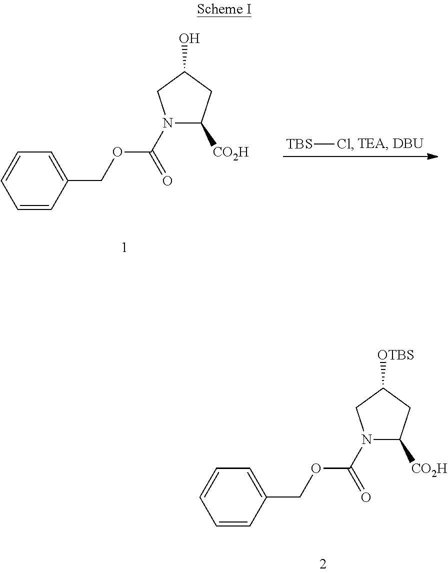

### 4-(tert-Butyl-dimethyl-silanyloxy)-pyrrolidine-1,2-dicarboxylic acid 1-benzyl ester (2)

A solution of Z-Hyp-OH (1, 300 g, 1.13 mol), TEA (395 mL, 2.83 mol), and DBU (17.2 g, 1.13 mol) in DMF (1.25 L) was stirred in a cold water bath while a suspension of TBS-Cl (188 g, 1.24 mol) in DMF (270 mL) was added slowly at 21-26° C. [Note: moderately exothermic]. The resulting thin suspension was stirred for 22 h at ambient temperature. The reaction mixture was cooled to 2° C. and quenched with water (1.54 L) at ≦26° C. [Note: the pH of the aqueous layer was 8.5-9.0]. MTBE (3 L) was added and the mixture was acidified to pH 3-4 with conc. HCl (168 g) at 17-19° C. The organic layer was separated and washed with water (2×1.5 L). The organic layer was concentrated in vacuo and dried by additional MTBE distillation. Toluene (2×500 mL) was added and distilled to remove moisture to provide 603 g of 2 as a light yellow-colored oil [Note: the water content by KF analysis was 508 ppm]. Based on drying a small sample of 2 to a solid, the contained weight of 2 was 412 g (96% yield, not corrected for purity).

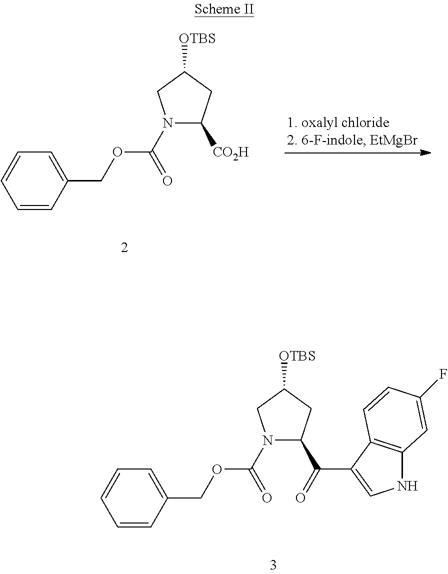 [SMILES: CC(C)(C)[Si](C)(C)O[C@@H]1C[C@@H](C(=O)O)N(C(=O)OCc2ccccc2)C1 ; CC(C)(C)[Si](C)(C)O[C@@H]1C[C@@H](C(=O)c2c[nH]c3cc(F)ccc23)N(C(=O)OCc2ccccc2)C1]

### 4-(tert-Butyl-dimethyl-silanyloxy)-2-(6-fluoro-1H-indole-3-carbonyl)-pyrrolidine-1-carboxylic acid benzyl ester (3)

Z-Hyp(OTBS)-OH (2, 55.5 g, 145 mmol) was dissolved in toluene (265 mL). DMF (0.1 mL) and oxalyl chloride (22.4 g, 174 mmol) were added at ambient temperature. After 2-3 h, the bubbling stopped. After 4 h, the mixture was concentrated in vacuo (65° C. bath, ca. 30 min) to provide 95 g of a light yellow-colored solution which was confirmed to be acid chloride by ¹H NMR analysis.

6-Fluoroindole (39.2 g, 290 mmol) was dissolved in anhydrous chlorobenzene (300 mL) and toluene (200 mL) and the solution was cooled to −4° C. using an ice/acetone bath. A solution of 3M EtMgBr in diethyl ether (101 g, 294 mmol) was added over 31 minutes at ≦2.5° C. resulting in a pale amber-colored solution. After 30 min, the acid chloride/toluene solution (vide supra) was added over 45 minutes at <2° C. The reaction mixture was kept cold for 1 h then allowed to slowly warm. After ca. 4 h (10.6° C.), the reaction mixture was quenched with glacial HOAc (9.0 g, exothermic to 17.5° C.) and then water (exothermic). Water (200 mL) and EtOAc (300 mL) were added and the organic layer was separated and washed with water (100 mL, slow separation). The organic layer was concentrated in vacuo to afford 227 g of 3 as an amber-colored oil which was used without further purification.

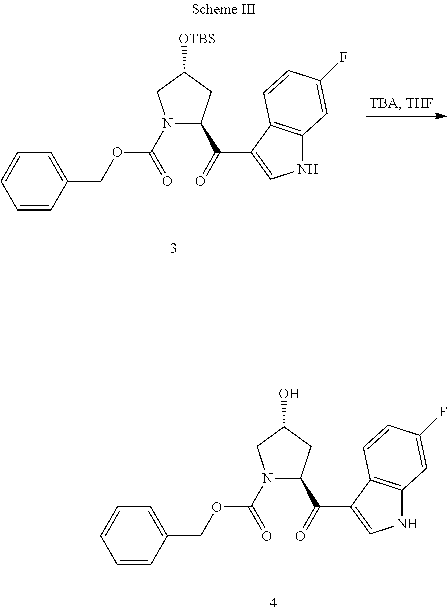 [SMILES: CC(C)(C)[Si](C)(C)O[C@@H]1C[C@@H](C(=O)c2c[nH]c3cc(F)ccc23)N(C(=O)OCc2ccccc2)C1 ; O=C(c1c[nH]c2cc(F)ccc12)[C@@H]1C[C@@H](O)CN1C(=O)OCc1ccccc1]

### 2-(6-Fluoro-1H-indole-3-carbonyl)-4-hydroxy-pyrrolidine-1-carboxylic acid benzyl ester (4)

To a solution containing 3 (227 g) in THF (600 mL) was added 1 M TBAF in THF (160 mL) at ambient temperature. After 9 h, another 20 mL of the 1 M TBAF/THF solution was added. After ca. 48 h, the reaction mixture was concentrated in vacuo and then redissolved in EtOAc (600 mL). The organic solution was washed with water (310 mL) and the product precipitated to form a thick suspension which was filtered (slow). The solids were washed with EtOAc (165 mL in portions) and dried to provide 43 g of 4. The combined filtrate was concentrated in vacuo to precipitate an additional 4.8 g of 4 after drying.

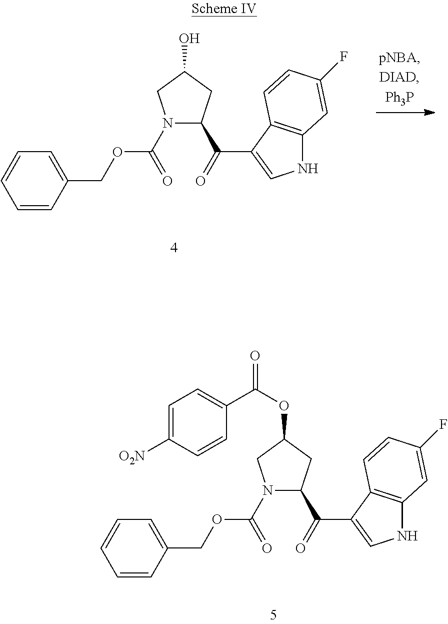 [SMILES: O=C(c1c[nH]c2cc(F)ccc12)[C@@H]1C[C@@H](O)CN1C(=O)OCc1ccccc1 ; O=C(O[C@H]1C[C@@H](C(=O)c2c[nH]c3cc(F)ccc23)N(C(=O)OCc2ccccc2)C1)c1ccc([N+](=O)[O-])cc1]

### 2-(6-Fluoro-1H-indole-3-carbonyl)-4-(4-nitro-benzoyloxy)-pyrrolidine-1-carboxylic acid benzyl ester (5)

A solution containing 4 (51.1 g, 134 mmol), 4-nitrobenzoic acid (27.9 g, 167 mmol) and triphenylphosphine (48.9 g, 187 mmol) in anhydrous THF (700 mL) and DMF (175 mL) was cooled to 2° C. DIAD (37.4 mL, 194 mmol) was added over 1 h at 2-3° C. After 1 h, the solution was allowed to warm to ambient temperature. After ca. 16 h, the reaction mixture was concentrated in vacuo and MeOH (250 mL) was added and concentrated to form a thick suspension (322 g).

Additional MeOH (250 mL) was added and the solution was concentrated in vacuo to afford a thick suspension (420 g) that was chilled in an ice bath. After ca. 1.5 h, the solid was collected on a vacuum filter and washed with chilled MeOH (190 mL). The product was air-dried on the filter to provide 82.9 g (>100%) of 5 as a light yellow-colored solid which was used directly in the next reaction.

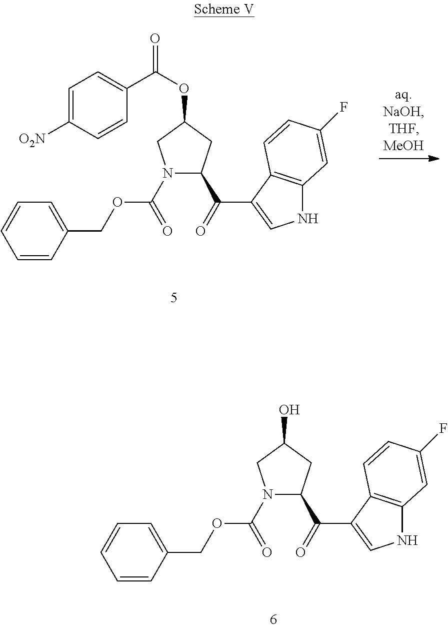 [SMILES: O=C(O[C@H]1C[C@@H](C(=O)c2c[nH]c3cc(F)ccc23)N(C(=O)OCc2ccccc2)C1)c1ccc([N+](=O)[O-])cc1 ; O=C(c1c[nH]c2cc(F)ccc12)[C@@H]1C[C@H](O)CN1C(=O)OCc1ccccc1]

### 2-(6-Fluoro-1H-indole-3-carbonyl)-4-hydroxy-pyrrolidine-1-carboxylic acid benzyl ester (6)

To a suspension of 5 (82.9 g) in THF (600 mL), MeOH (200 mL), and water (100 mL) was added 50% aq. NaOH (16.0 g, 200 mmol) [Note: exothermic; temp. increase: 23.7° C. to 25.9° C.]. After 2 h, glacial HOAc (5.3 g) was added to adjust the pH to 7-8 [Note: the orange-colored solution changed to pale yellow] and the reaction mixture was concentrated in vacuo. Water (500 mL) was added and solvent was removed in vacuo until a thick suspension formed. The solid was collected on a vacuum filter and washed with water (400 mL in portions). The solid was dried in a vacuum oven at 55° C. to afford 42.6 g (83%, 2 steps) of 6 as an off-white solid.

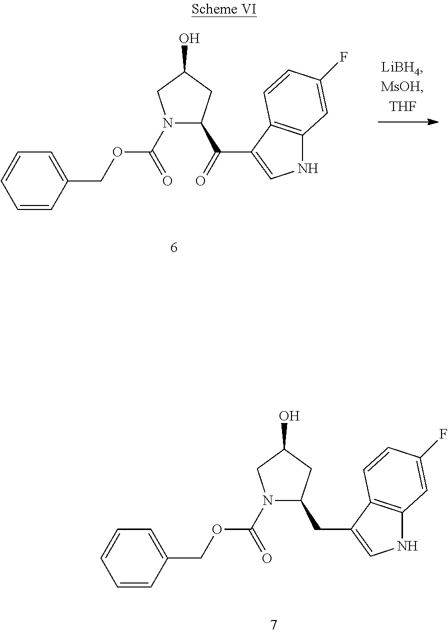 [SMILES: O=C(c1c[nH]c2cc(F)ccc12)[C@@H]1C[C@H](O)CN1C(=O)OCc1ccccc1 ; O=C(OCc1ccccc1)N1C[C@@H](O)C[C@H]1Cc1c[nH]c2cc(F)ccc12]

### 2-(6-Fluoro-1H-indol-3-ylmethyl)-4-hydroxy-pyrrolidine-1-carboxylic acid benzyl ester (7)

To a suspension of 6 (10.1 g, 26 mmol) in anhydrous THF (200 mL) was added 2M LiBH₄ in THF (26.2 mL, 52 mmol) over ca. 7 min [Note: exothermic; temp. increase: 21.5° C. to 28.2° C.]. After 2.5 h, the pale, yellow-colored solution was cooled to ca. 11° C. and methanesulfonic acid (4.66 g, 48 mmol) was added over ca. 4 min [Note: exothermic; temp. increase to 14.2° C.].

After 16 h, the reaction mixture was cooled in an ice-bath and carefully quenched with water (50 mL) [Note: the addition of water was exothermic and released a large quantity of gas]. Following the addition of water, the pH was adjusted to 1 with conc. HCl (1.9 g). The reaction mixture was concentrated to remove THF and the aqueous solution was extracted with EtOAc (110 mL). The organic layer was separated and washed with water (2×50 mL) [Note: final pH about 5]. The organic solution was concentrated in vacuo and azeotropically dried using anhydrous EtOAc to provide 10.2 g of 7 as a white foam [Note: 87.7 A % by HPLC analysis].

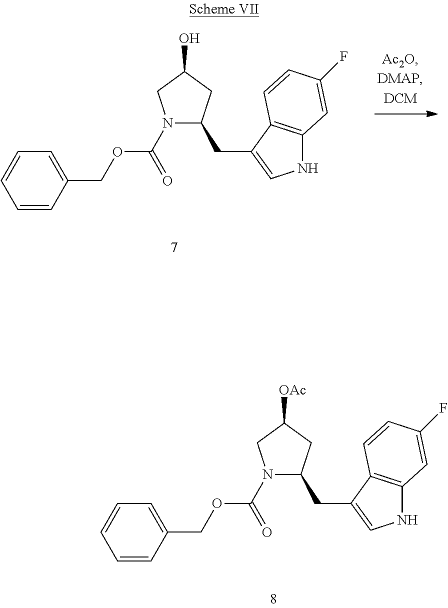 [SMILES: O=C(OCc1ccccc1)N1C[C@@H](O)C[C@H]1Cc1c[nH]c2cc(F)ccc12 ; CC(=O)O[C@H]1C[C@@H](Cc2c[nH]c3cc(F)ccc23)N(C(=O)OCc2ccccc2)C1]

### 4-Acetoxy-2-(6-fluoro-1H-indol-3-ylmethyl)-pyrrolidine-1-carboxylic acid benzyl ester (8)

To a solution containing 7 (4.7 g, 12.8 mmol) and DMAP (81 mg, 0.66 mmol) in DCM (100 mL) was added acetic anhydride (2.6 g, 25.5 mmol) at ambient temperature. After 16 h, the reaction mixture was quenched with a MeOH (ca. 3 mL) and washed successively with 10% aq. Na₂CO₃ (50 mL), dilute HCl (50 mL), and 10% aq. Na₂CO₃ (50 mL). The organic solution was concentrated in vacuo and filtered through a short column of silica gel (ca. 25 g) [eluant: DCM (200 mL) to 0.5% (v/v) MeOH/DCM (80 mL) to 2% MeOH/DCM (100 mL) to 5% MeOH/DCM (100 mL)]. The product-containing fractions were combined and concentrated to provide 3.28 g (63%) of 8 as a white foam [Note: 94.3 A % by HPLC analysis].

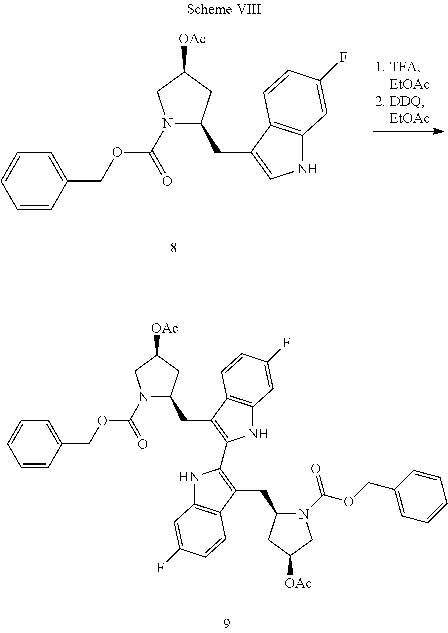 [SMILES: CC(=O)O[C@H]1C[C@@H](Cc2c[nH]c3cc(F)ccc23)N(C(=O)OCc2ccccc2)C1 ; CC(=O)O[C@H]1C[C@@H](Cc2c(-c3[nH]c4cc(F)ccc4c3C[C@@H]3C[C@H](OC(C)=O)CN3C(=O)OCc3ccccc3)[nH]c3cc(F)ccc23)N(C(=O)OCc2ccccc2)C1]

### 4-Acetoxy-2-[3′-(4-acetoxy-1-benzyloxycarbonyl-pyrrolidin-2-ylmethyl)-6,6′-difluoro-1H,1′H-[2,2′]biindolyl-3-ylmethyl]-pyrrolidine-1-carboxylic acid benzyl ester (9)

A solution containing 8 (2.9 g, 7.1 mmol) in EtOAc (ca. 5 mL) was cooled in an ice-bath and pre-cooled TFA (20.3 mL) was added in one portion. The resulting yellow-colored solution was stirred at 2-4° C. After 4.75 h, the cold reaction mixture was transferred (via canula) with stirring into a pre-cooled mixture of EtOAc (30 mL), and 25% aq. K₂CO₃ (80.7 g). The aqueous layer was separated and extracted with EtOAc (3×30 mL) and the combined organic extracts were washed with 10% aq. Na₂CO₃ (30 g). The organic solution was concentrated in vacuo and azeotropically dried using anhydrous EtOAc to afford 2.95 g of indolylindoline diastereomers as a yellow-colored foam which was used directly in the next reaction. Mass spectrum (ESI), m/z 821.3 [(M)+; calcd for C₄₆H₄₆F₂N₄O₈: 820.9].

To a solution containing the indolylindoline diastereomers (2.95 g) in EtOAc (30 mL) was added DDQ (885 mg, 3.9 mmol) in one portion [Note: exothermic; temp. increase: 26° C. to 31.6° C.]. After 3 h, the dark orange/brown-colored reaction mixture was filtered through Celite® which was subsequently rinsed with EtOAc (50 mL). [Note: a second reaction performed at 0.5 mmol-scale was combined for work-up]. The filtrate was washed with 10% aq. Na₂CO₃ (2 washes: 74 g, then 58 g). The organic layer was concentrated in vacuo to provide 2.14 g of 9 as a light brown-colored solid.

The Celite® pad was further rinsed with THF (100 mL) which was concentrated in vacuo to provide another 1.12 g of 9 as a beige-colored solid. The combined solids were dissolved in isopropyl acetate (iPrAc, 50 mL). The iPrAc solution was reduced to ca. 20 mL and resulting suspension was warmed to reflux, cooled to ambient temperature, and then placed in an ice-bath. After 1 h, the solid was collected by vacuum filtration, washed with iPrAc (10 mL) and dried in a vacuum oven to afford 2.13 g (65%, 2 steps) of 9 as a beige-colored solid [Note: ˜100 A % by HPLC analysis].

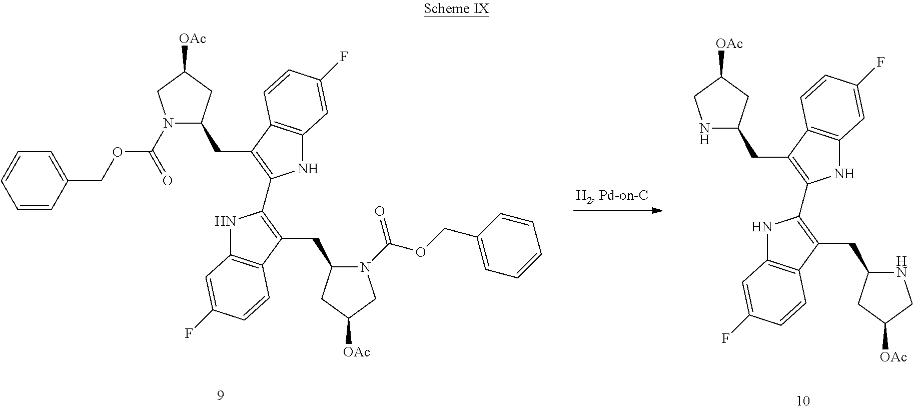 [SMILES: CC(=O)O[C@H]1C[C@@H](Cc2c(-c3[nH]c4cc(F)ccc4c3C[C@@H]3C[C@H](OC(C)=O)CN3C(=O)OCc3ccccc3)[nH]c3cc(F)ccc23)N(C(=O)OCc2ccccc2)C1 ; CC(=O)O[C@@H]1CN[C@H](Cc2c(-c3[nH]c4cc(F)ccc4c3C[C@@H]3C[C@H](OC(C)=O)CN3)[nH]c3cc(F)ccc23)C1]

### Acetic acid 5-[3′-(4-acetoxy-pyrrolidin-2-ylmethyl)-6,6′-difluoro-1H,1′H-[2,2′]biindolyl-3-ylmethyl]-pyrrolidin-3-yl ester (10)

A suspension containing 9 (35 g, 42.7 mmol) in 1:1 EtOAc/MeOH (400 mL) was distributed into two 500 mL Parr bottles (ca. 200 mL/each), and charged with 10% Pd-on-C (wet, 5000 mg/each, Aldrich®). The reaction mixture was pressurized to 50 PSI H₂ and shaken for 3 h. The reaction mixture was filtered through a pad of Celite® and the solids were washed with EtOAc. The clarified filtrate was concentrated in vacuo to afford 24 g of 10 as an off-white solid which was used directly in the next reaction.

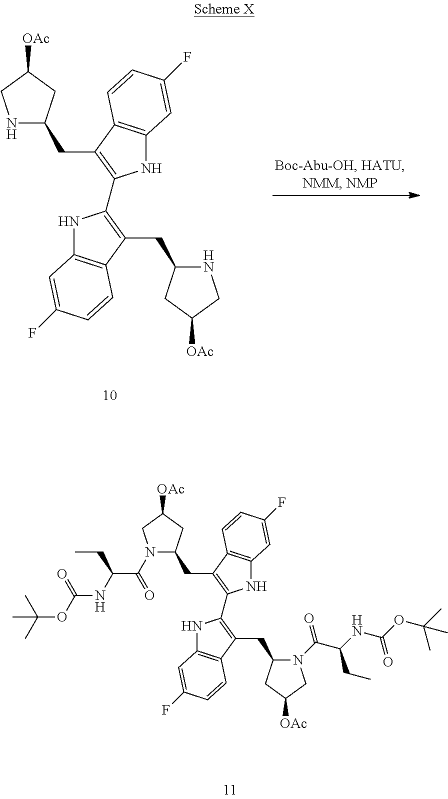 [SMILES: CC(=O)O[C@@H]1CN[C@H](Cc2c(-c3[nH]c4cc(F)ccc4c3C[C@@H]3C[C@H](OC(C)=O)CN3)[nH]c3cc(F)ccc23)C1 ; CC[C@H](NC(=O)OC(C)(C)C)C(=O)N1C[C@@H](OC(C)=O)C[C@H]1Cc1c(-c2[nH]c3cc(F)ccc3c2C[C@@H]2C[C@H](OC(C)=O)CN2C(=O)[C@H](CC)NC(=O)OC(C)(C)C)[nH]c2cc(F)ccc12]

### Acetic acid 5-{3′-[4-acetoxy-1-(2-tert-butoxycarbonylamino-butyryl)-pyrrolidin-2-ylmethyl]-6,6′-difluoro-1H,1′H-[2,2′]biindolyl-3-ylmethyl}-1-(2-tert-butoxycarbonylamino-butyryl)-pyrrolidin-3-yl ester (11)

To a solution containing Boc-Abu-OH (20.4 g, 100 mmol) and HATU (42.0 g, 110 mmol) in anhydrous NMP (150 mL) at 0° C. was added NMM (16 mL, 150 mmol) followed by a solution of 10 (24 g, 42 mmol) in NMP (100 mL). The reaction mixture was slowly warmed to ambient temperature. After 16 h, the reaction mixture was diluted with MTBE (1000 mL) and the heterogeneous mixture was washed with water (500 mL). The layers were separated and the organic phase formed a heterogeneous suspension. MTBE (1000 mL) and EtOAc (500 mL) were added and the now-homogeneous solution was washed successively with 1 N HCl (2×100 mL), saturated aqueous NaHCO₃ (2×100 mL), brine, dried over anhydrous Na₂SO₄, filtered, and concentrated. The residue was dissolved in 1:1 DCM/MeOH (600 mL) and DCM (ca. 200 mL) was removed via distillation at 50° C. [Note: a small quantity of white precipitate was observed]. MeOH (200 mL) was added and additional solvent was removed (ca. 200 mL) at 50° C. The heterogeneous mixture was cooled at −5° C. After 16 h, the solid was collected by vacuum filtration and washed with cold MeOH. The solid was dried under high vacuum to afford 32 g of 11 as an off-white solid.

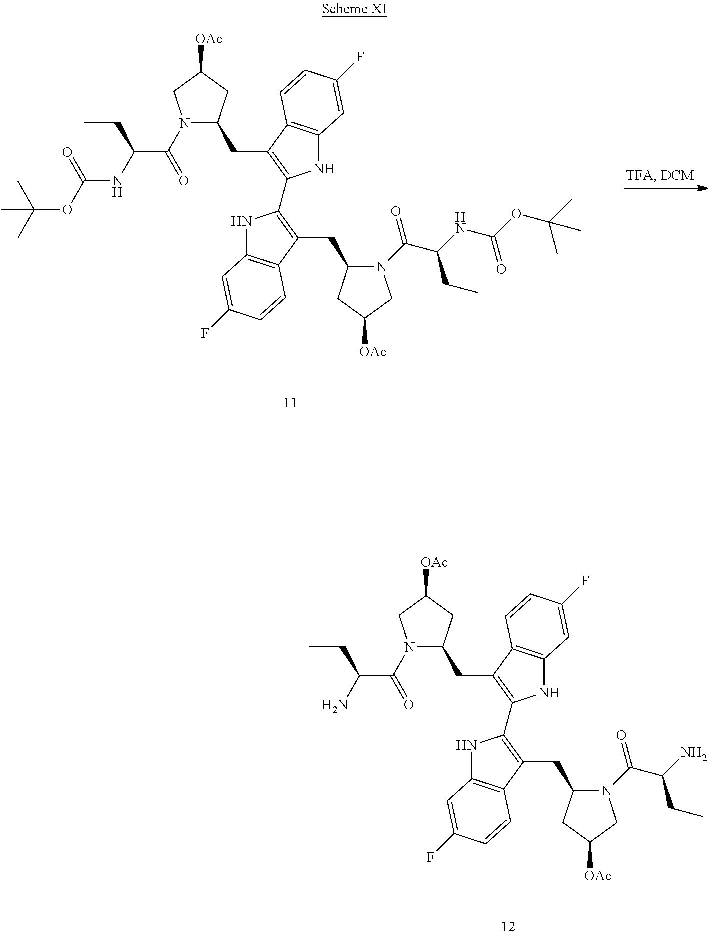

### Acetic acid 5-{3′-[4-acetoxy-1-(2-amino-butyryl)-pyrrolidin-2-ylmethyl]-6,6′-difluoro-1H,1′H-[2,2′]biindolyl-3-ylmethyl}-1-(2-amino-butyryl)-pyrrolidin-3-yl ester (12)

A solution containing 11 (27.5 g, 30 mmol) in DCM (200 mL) was cooled to 0° C. TFA (50 mL) was added and the reaction was monitored by LC/MS analysis until complete conversion of 11 to 12 (ca. 3 h). The solvent was removed in vacuo and the dark, green-colored residue was dissolved in EtOAc (ca. 1 L). The EtOAc solution was carefully poured into a saturated aqueous NaHCO₃/ice/water mixture to neutralize the residual TFA. The organic phase was separated and washed twice with saturated aqueous NaHCO₃ then once with brine. The combined aqueous washes were back-extracted with EtOAc (2×100 mL) and the combined organic extracts were dried over anhydrous Na₂SO₄, filtered, and concentrated to afford 22 g of crude 12 as an off-white solid.

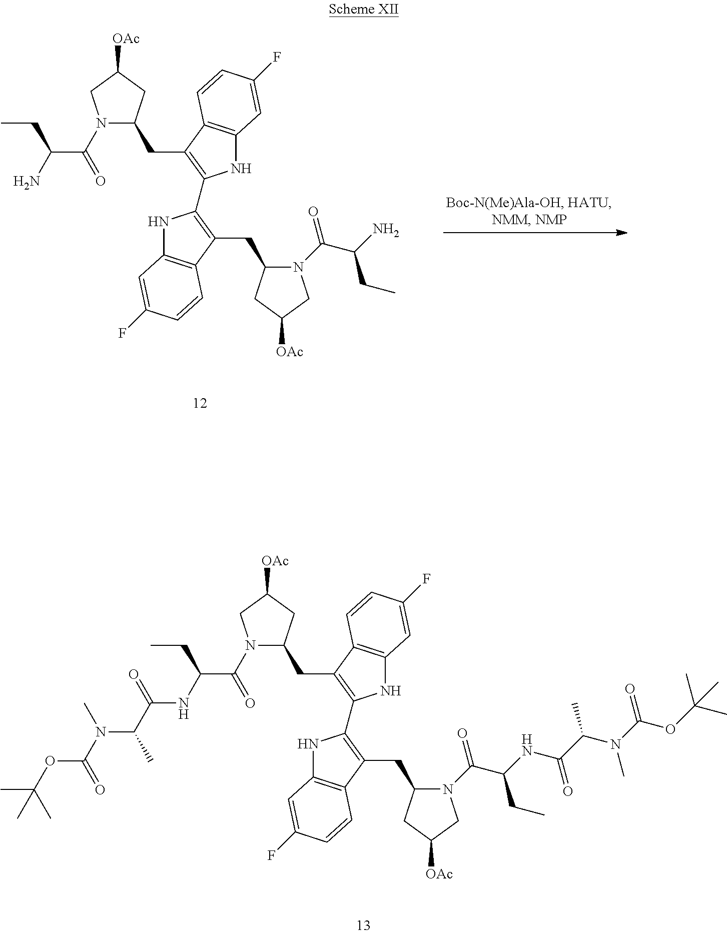 [SMILES: CC[C@H](N)C(=O)N1C[C@@H](OC(C)=O)C[C@H]1Cc1c(-c2[nH]c3cc(F)ccc3c2C[C@@H]2C[C@H](OC(C)=O)CN2C(=O)[C@@H](N)CC)[nH]c2cc(F)ccc12 ; CC[C@H](NC(=O)[C@H](C)N(C)C(=O)OC(C)(C)C)C(=O)N1C[C@@H](OC(C)=O)C[C@H]1Cc1c(-c2[nH]c3cc(F)ccc3c2C[C@@H]2C[C@H](OC(C)=O)CN2C(=O)[C@H](CC)NC(=O)[C@H](C)N(C)C(=O)OC(C)(C)C)[nH]c2cc(F)ccc12]

### Acetic acid 5-(3′-{4-acetoxy-1-[2-(2-methyl-(tert-butoxycarbonyl)-amino-propionylamino)-butyryl]-pyrrolidin-2-ylmethyl}-6,6′-difluoro-1H,1′H-[2,2′]biindolyl-3-ylmethyl)-1-[2-(2-methyl-(tert-butoxycarbonyl)-amino-propionylamino)-butyryl]-pyrrolidin-3-yl ester (13)

To a solution containing Boc-N(Me)Ala-OH (14.6 g, 72 mmol) and HATU (30.4 g, 80 mmol) in anhydrous NMP (150 mL) at 0° C. was added NMM (12 mL, 105 mmol) followed by addition of 12 (30 mmol) in NMP (200 mL). The resulting mixture was allowed to warm to ambient temperature. After 16 h, the reaction mixture was diluted with diethyl ether (1 L) and washed successively with water (1 L), 1N HCl (2×100 mL), saturated aqueous NaHCO₃ (2×100 mL), brine, dried over anhydrous Na₂SO₄, filtered, concentrated to afford 33.5 g of crude 13.

The crude 13 was dissolved in EtOH (50 mL) and then slowly added to water (1000 mL) with vigorous stirring at 50° C. which resulted in the precipitation of a white solid. The heterogeneous mixture was cooled to −5° C. After 16 h, the solid was collected by vacuum filtration and washed with water. The wet solid was dried under high vacuum at 50° C. to afford 29.9 g of 13 as an off-white solid.

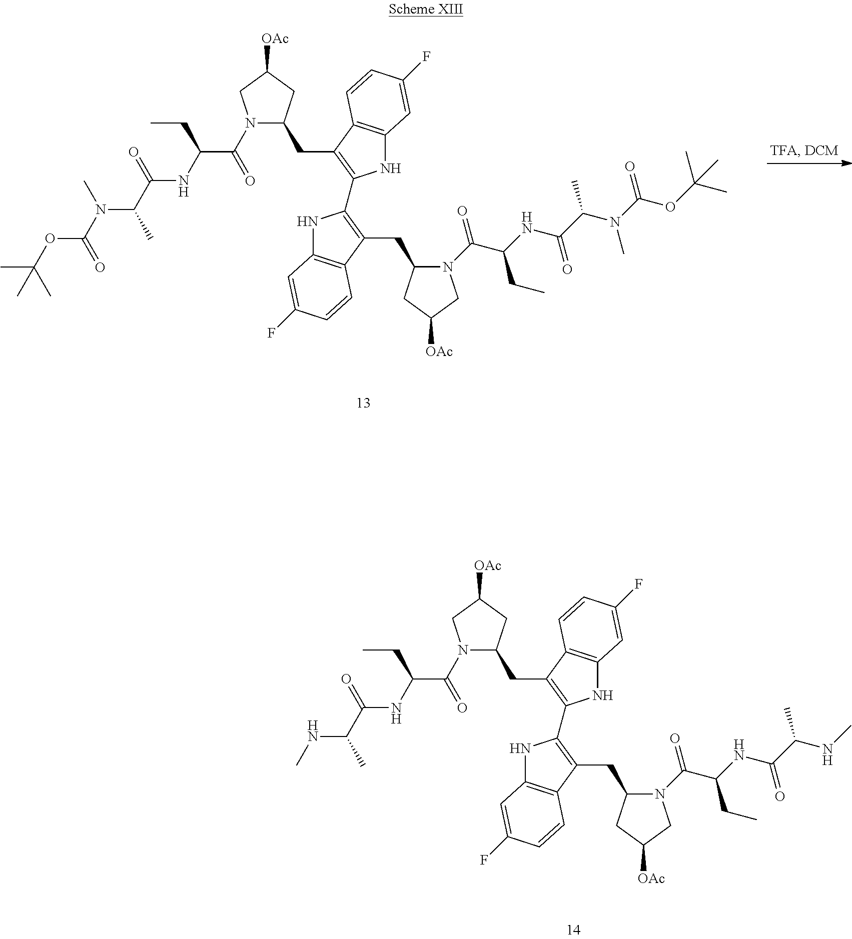

### Acetic acid 5-(3′-{4-acetoxy-1-[2-(2-methylamino-propionylamino)-butyryl]-pyrrolidin-2-ylmethyl}-6,6′-difluoro-1H,1′H-[2,2′]biindolyl-3-ylmethyl)-1-[2-2-methylamino-propionylamino)-butyryl]-pyrrolidin-3-yl ester (14)

A solution containing 13 (28.5 g, 26 mmol) in DCM (150 mL) was cooled to 0° C. TFA (50 mL) was added. After 30 min, the reaction mixture was warmed to ambient temperature and monitored until LC/MS analysis revealed complete conversion of 13 to 14 (ca. 4 h). The solvent was removed in vacuo and the dark, green-colored residue was dissolved in EtOAc (500 mL) and carefully poured onto an aqueous NaHCO₃/ice mixture. The aqueous phase was separated and back-extracted with EtOAc (2×250 mL). The combined organic extracts were washed several times with saturated aqueous NaHCO₃, then brine, dried over anhydrous Na₂SO₄, filtered, and concentrated to afford 24 g of 14 as a light yellow-colored solid.

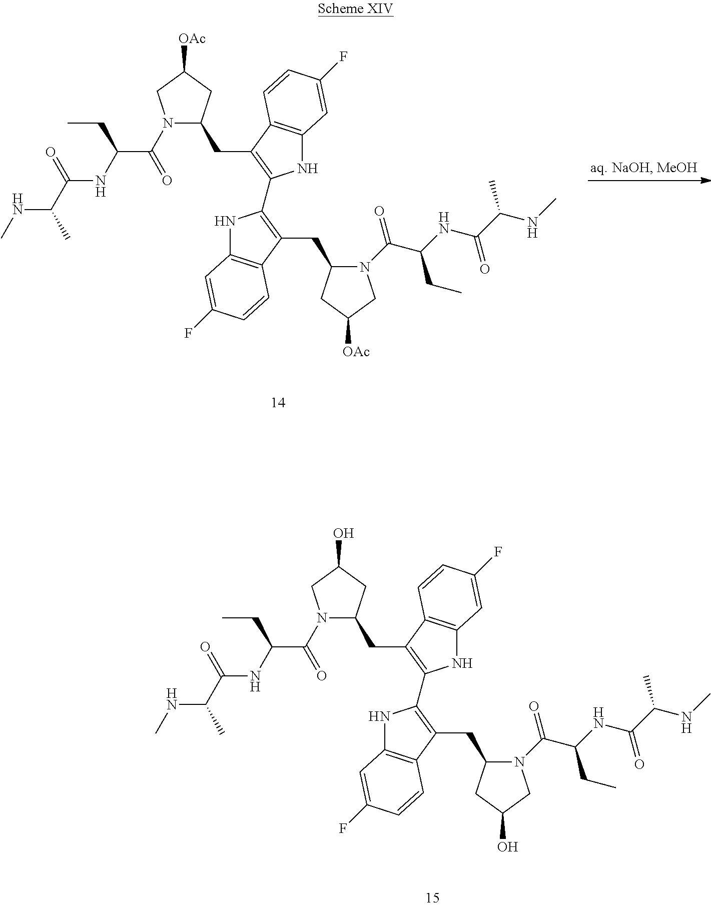

### N-{1S-[2R-(6,6′-Difluoro-3′-{4S-hydroxy-1-[2S-(2S-methylamino-propionylamino)-butyryl]-pyrrolidin-2R-ylmethyl}-1H,1′H-[2,2′]biindolyl-3-ylmethyl)-4S-hydroxy-pyrrolidine-1-carbonyl]-propyl}-2S-methylamino-propionamide (15)

To a solution containing 14 (24 g) in MeOH (200 mL) was added 1 M NaOH (80 mL) at 0° C. The reaction mixture was degassed and maintained under a nitrogen atmosphere wrapped with aluminum foil. The ice-bath was removed. After 60 min, the MeOH was removed in vacuo and the residue was diluted with water (200 mL) and extracted with EtOAc (500 mL). The aqueous phase was separated and back-extracted with EtOAc (2×150 mL). The combined organic extracts were washed with brine and dried over anhydrous Na₂SO₄, filtered, and concentrated to afford 22.5 g of crude 15 as a light, brown/yellow-colored solid.

The crude 15 (22.5 g) was dissolved in MeOH (50 mL) and EtOAc (200 mL). The volume was reduced (50%) by distillation at reduced pressure at 60° C. using a rotary evaporator. MTBE (300 mL) was added and the cloudy solution was warmed to 60° C. After 30 min, the solution was cooled to ambient temperature and then maintained at −5° C.

After 16 h, the solid was collected by vacuum filtration and washed with cold 25% EtOAc/MTBE and dried under high vacuum at ambient temperature to afford 16.6 g of 15 as an off-white solid. An additional 5.5 g of 15 was recovered from the filtrate via solvent removal and vacuum drying. ¹H NMR (300 MHz, CDCl₃): δ11.74 (s, 2H), 8.27 (d, J=8.7 Hz, 2H), 7.71 (dd, J=5.4, 8.4 Hz, 2H), 7.55 (dd, J=2.4, 9.6 Hz, 2H), 6.88 (ddd, J=2.4, 9.3, 9.3 Hz, 2H), 4.62-4.78 (m, 4H), 4.43 (dd, J=9.3, 9.9 Hz, 2H), 4.03 (dd, J=4.8, 11.4 Hz, 2H), 3.80 (d, J=11.4 Hz, 2H), 3.66 (dd, J=2.7, 14.4 Hz, 2H), 3.53 (dd, J=11.4, 14.4 Hz, 2H), 3.11 (q, J=6.9 Hz, 2H), 2.56 (s, 6H), 2.45 (m, 2H), 2.19 (d, J=14.4 Hz, 2H), 1.76-2.10 (m, 6H), 1.59 (br s, 2H), 1.39 (d, J=6.9 Hz, 6H), 1.22-1.38 (m, 2H), 1.07 (t, J=7.2 Hz, 6H) ppm; ¹³C NMR (75 MHz, d₆-DMSO): δ175.2, 172.8, 161.6, 158.5, 137.3, 137.2, 128.4, 128.3, 126.4, 120.8, 120.6, 109.4, 108.7, 108.4, 98.4, 98.0, 70.8, 60.2, 59.9, 56.6, 51.8, 36.4, 35.3, 28.3, 25.6, 20.0, 10.6 ppm. Mass spectrum (ESI), m/z 807.5 [(M)+; calcd for C₄₂H₅₆F₂N₈O₆: 806.9].

Data from various experiments with Compound 15 (i.e., TL32711 also known as birinapant) are provided in the following Examples.

### Example 2

### Dose Scheduling and Efficacy Analysis of the SMAC Mimetic TL32711 in Primary Melanoma Tumor Xenotransplant Models

Initial pharmacokinetics modeling of TL32711 in mice bearing the MDA-MB-231 tumor indicated a potential efficacy benefit may be possible with a biweekly dosing schedule. The objectives of the current study were to 1) evaluate the efficacy of TL32711 as a single agent in primary human melanoma tumor xenograft models, 2) assess the efficacy and tolerability of TL32711 in combination with carboplatin and paclitaxel and 3) determine if a biweekly dosing schedule is more effective than weekly administration.

Significant tumor growth inhibition was observed in 5 of 6 of the primary melanoma tumor xenografts evaluated following treatment with single agent TL32711 (30 mg/kg IP). Combining TL32711 with carboplatin and paclitaxel resulted in a further enhancement in anti-tumor efficacy with tumor regressions noted in 4 of the 6 models without any marked changes in tolerability (<14% reduction in bodyweight). Based on the initial PK modeling a follow up study was conducted to assess the activity of TL32711 in a primary melanoma model when the dose was fractionated (15 mg/kg twice/week versus 30 mg/kg once/week). Surprisingly, the biweekly dosing schedule did not result in enhanced anti-tumor activity and demonstrated equivalent suppression of cIAP1 in tumors compared to the weekly dosing schedule.

Pharmacokinetic analysis of the TL32711 in tumor tissue at 15, 30 and 60 mg/kg revealed that TL32711 exhibits a greater than dose proportional relationship in that a 4-fold increase in dose, resulted in a 14-fold increase in exposure. This increase in exposure led to a change in the TL32711 tumor half-life from 56 to 166 hrs, possibly due to the saturation of an efflux transporter at higher dose levels.

Together, these data show that TL32711 is highly active in primary human melanoma xenografts and that efficacy can be enhanced by combination therapy with carboplatin and paclitaxel without reducing tolerability. These data also demonstrate that biweekly dosing confers no advantage over the current clinical weekly dosing regimen due to the dose dependent changes in TL32711 half-life and exposure observed in tumor tissue.

### Example 3

### Phase 1 PK/PD Analysis of the Smac Mimetic TL32711 Demonstrates Potent and Sustained cIAP1 Suppression in Patient PBMCs and Tumor Biopsies

The pharmacokinetics (PK) and pharmacodynamics (PD) of TL32711 have been studied in human tumor xenografts, patient plasma/PBMCs and Phase 1 tumor biopsy samples. In mice bearing the MDA-MB-231 xenograft, TL32711 is rapidly and extensively taken up into the tumor (tumor/plasma AUC ratio>22) and is eliminated slowly with a half-life of 96 hrs (20 hrs in plasma). A PK/PD link model was used to characterize the relationship between TL32711 tumor concentrations and cIAP1 suppression. cIAP1 suppression was dose and time dependent with cIAP1 levels reduced to <20% baseline within 30 minutes and with >70% inhibition maintained 7-14 days post treatment following a single IV bolus dose (5 mg/kg). TL32711 had a potent effect on tumor cIAP1 levels (EC50 24 ng/g) and caused significant tumor growth inhibition and regressions at doses ≧2.5 mg/kg q3D. Efficacy has also been evaluated in primary human melanoma tumors, recently derived from patients and transplanted into nude mice. Significant tumor growth inhibition was observed in 5/6 primary melanoma tumor xenografts with mean Day 7 tumor concentrations of 187, 579 and 2658 ng/g at 15, 30 and 60 mg/kg respectively. TL32711 PK/PD (drug concentration analysis and cIAP1 degradation in PBMCs and tumor biopsies) has also been investigated in patients as part of the single agent Phase I study. Following weekly, 30 min IV infusions TL32711 plasma PK was dose proportional and non-accumulating (0.18 to 47 mg/m2). Plasma PK was tri-exponential with a long terminal t½ (73-79 hrs). The target AUC in plasma for therapeutic activity (71 h·ng/mL) based on the MDA-MB-231 model was achieved in patients at dose >2.88 mg/m2 (Mean AUC 86 h·ng/mL). This exposure was associated with marked uptake and retention in PBMCs (t_{1/2}=29-35 hrs) and resulted in prolonged cIAP1 suppression over 7 days. A dose related increase in PBMC PARD cleavage and plasma caspase-3 activity was also observed indicative of apoptosis pathway activation. TL32711 PK/PD was also assessed in tumor biopsy samples from patients 4 hrs to 6 days post treatment (11.5 to 17.2 mg/m²). TL32711 is extensively taken up into the tumor with levels >350 ng/g on day 6, significantly in excess of the EC₅₀ for cIAP1 inhibition. Estimated tumor exposure at 35 to 47 mg/m² was also in excess of the measured drug levels observed at 15 to 30 mg/kg in the primary human tumor xenograft models in mice. Together these PK/PD data show that TL32711 results in potent and sustained cIAP1 suppression over 7 days at tolerable dose levels with evidence of apoptosis pathway activation and promising early signs of anti-tumor activity in patients. Selected results and conclusions of these studies are summarized in the following list:
1) To date, TL32711 has been well tolerated in patients and Phase 1 dose escalation continues to define the single agent maximum tolerated dose (MTD).
2) TL32711 is rapidly taken up into tumor tissue with a long terminal half-life of 96 hrs (MDA-MB-231 xenograft) or 52 hrs (human tumor biopsies).
3) TL32711 rapidly (within 4 hrs) and potently inhibits cIAP1 in MDA-MB-231 tumor tissue (IC50 24 ng/g; IC75 135 ng/g) in a dose dependent manner.
4) PK/PD analyses in mice indicated that tumor tissue was approximately 2× to 100× more sensitive to the cIAP1 inhibition compared to other normal tissues.
5) Significant tumor growth delay and regressions were observed when cIAP1 levels in tumors was inhibited by >75% throughout the dosing interval in mice bearing the MDA-MB-231 xenograft.
6) TL32711 PK was dose proportional over the dose range 0.18 to 47 mg/m² in Phase 1 patients.
7) The PK/PD response in patient biopsies and PBMCs were very similar to the response observed in the MDA-MB-231 xenograft.
8) PK/PD modeling of the cIAP1 response in patients indicates that the current dose level of 47 mg/m² results in >75% cIAP1 inhibition throughout the weekly dosing interval.
9) In summary, TL32711 causes potent and sustained cIAP1 suppression over 7 days at tolerable dose levels, apoptosis pathway activation and promising early signs of anti-tumor activity in patients.

### Example 4

### Phase 1 Study of the Smac Mimetic TL32711 in Adult Subjects with Advanced Solid Tumors & Lymphoma to Evaluate Safety, Pharmacokinetics, Pharmacodynamics and Anti-Tumor Activity

A clinical study was conducted having the following primary objective: To determine the maximum tolerated dose and characterize the safety and tolerability of TL32711 when administered as a 30 minute intravenous infusion once weekly for 3 consecutive weeks followed by one week off (Cycle) repeated every 4 weeks as tolerated in patients with refractory solid tumors or lymphoma. The secondary objective was to assess the pharmacokinetics, pharmacodynamic effects and anti-tumor activity of TL32711.

Relevant information pertaining to the design of the clinical study is summarized in Tables 2-4.

TABLE 2
Eligibility

Inclusion Criteria:

Confirmed advanced metastatic or unresectable malignancy that is
refractory to currently available standard therapies
ECOG performance status of ≦2; life expectancy >3 mo
Adequate renal, hepatic and bone marrow function

Exclusion Criteria:

Received standard or investigational anti-cancer therapy within 4
weeks prior to first dose of TL32711
Symptomatic or uncontrolled brain metastases requiring current
treatment
Clinically significant auto-immune, cardiac or pulmonary disease

TABLE 3
Trial Design

Phase 1, multi-centered, open-label, dose-escalation 3 + 3 design, with
dose expansion at recommended Phase 2 dose
Dose levels escalated by 100%, If CTCAE v.4 drug-related AE Grade ≧2
or >1 change above baseline, subsequent cohorts escalated by 50%
or 33%
TL32711 administered as a 30 min IV infusion once weekly for 3
consecutive weeks followed by one week off (Cycle) repeated every 4
weeks IV until progression/toxicity/voluntary withdrawal.
Weekly study assessments (+C1D2, C1D16) until treatment discontinued

PK/PD markers (IAPs, apoptosis activation) - pre-dose and 4 and
24 hours post dose on Day 1 and 15, and pre-dose and 4 hours
post-dose on Day 8 dose
Restaging was done at the end of Cycle 2

TABLE 4
Patient Characteristics

| | |
|---|---|
| Patients Treated | 24 |
| (Cohorts 1-8) | |
| Median Age, yrs | 56.5 |
| (range) | (31-80 yrs) |
| Gender, n (%) | |
| Male | 15 (62.5%) |
| Female | 9 (37.5%) |
| ECOG | |
| Performance | |
| Status, n (%) | |
| 0 | 13 (54%) |
| 1 | 11 (46%) |
| 2 | 0 (0%) |

| Cancer Type | n | % |
|---|---|---|
| Anal | 1 | 4% |
| Appendiceal | 1 | 4% |
| Colon | 5 | 21% |
| Gastric | 2 | 8% |
| Head & Neck | 3 | 12% |
| Hodgkin's | 2 | 8% |
| Lymphoma | | |
| Melanoma | 3 | 13% |
| Ovarian | 2 | 8% |
| Pancreatic | 2 | 8% |
| Sarcoma | 2 | 8% |
| SCLC | 1 | 4% |

Safety and Anti-tumor activity results are summarized in Tables 5-6.

TABLE 5
Safety Summary
No Grade 3 or Grade 4 Adverse Events attributed to study drug
Most Common Drug-Related Adverse Events with incidence ≧2

| Adverse Event | Number of Grade 1 or 2 Events (%) |
|---|---|
| Nausea | 5 (14%) |
| Fever | 4 (11%) |
| Rash | 3 (8%) |
| Lymphocytopenia | 2 (6%) |

TABLE 6
Anti-Tumor Activity

CRC Patient 01-202 (Dose cohort 0.36 mg/m²)

Patient with relapsed progressive disease after 7 prior regimens
CT scan; 3 of 5 metastatic lesions decreased after 2 cycles of
TL32711 - Stable Disease by RECIST criteria
Patient received 6 cycles (24 weeks) of TL32711 before disease
progression

Melanoma Patient 01-703 (Dose cohort 11.5 mg/m²)

Patient with rapidly progressive disease prior to study
Stable Disease by RECIST criteria after 2 cycles of TL32711
Progressed after 3^{rd} cycle with increasing cutaneous lesions

CRC Patient 01-801 (Dose cohort 17.2 mg/m²)

Patient with progressive disease after multiple prior therapies
Patient's CEA decreased (150 to 90) and developed a 4-5 cm
photopenic lesion in a hepatic metastasis after 1 cycle of TL32711.
Patient had marked clinical improvement of early satiety and pain
during 1^{st} 2 cycles.
Patient progressed with development of a new liver lesion after
2 cycles

The following conclusions were drawn from this study:
1) TL32711 is well tolerated in patients with solid tumors and lymphoma with no dose-limiting toxicities and the MTD has not been reached.
2) TL32711 displays dose proportional PK, moderate to low inter-patient variability in Cmax and AUC, and a long terminal half-life in plasma (35 hours) with high uptake and retention in tumor tissues (49 hours).
3) TL32711 causes rapid (within 4 hours) and sustained (for 7 days) suppression of cIAP1 that is dose-dependent as measured in both PBMCs and tumor biopsies.
4) TL32711 causes dose-related activated serum caspase-3/7 and cleaved cytokeratin-18 levels.
5) Evidence of anti-tumor activity observed.

### Example 5

### Anti-Tumor Efficacy in Primary Pancreatic Adenocarcinoma Model

Pancreatic cancer is highly resistant to chemotherapeutic drugs and radiation. Inhibitors of apoptosis (IAPs) were overexpressed in pancreatic cancer cells and IAPs downregulation were shown to induce sensitivity to death receptor signaling, cytotoxic agents and radiation. A study was conducted to investigate the efficacy of TL32711 using a patient-derived primary pancreatic cancer explant model that mirrors the disease's biological heterogeneity.

Methods.

Effect of TL32711 alone and with TRAIL was evaluated in Panc1 by immunoblotting and Trypan blue staining Dose escalation studies were performed in 2 primary pancreatic tumors at i.p. 30 mg/kg, 45 mg/kg and 60 mg/kg every twice weekly and tumor volume were measured for 28 days. No significant toxicity was observed in the tumor-bearing mice at all dose levels. An additional 6 primary pancreatic tumors were evaluated at 60 mg/kg. H&E slides of donor patients for these tumors were evaluated and untreated tumors were analyzed by gene microarrays to explore for potential efficacy biomarkers. Tumor, plasma and liver samples were obtained from the dose escalation studies for pharmacokinetic analysis.

Results.

TL32711 treatment resulted in rapid cIAP1 degradation leading to caspase-3 activation in Panc1, and exerted a dose-dependent pro-apoptotic effect that was synergized with TRAIL co-incubation in in vitro studies. In primary tumor explant studies, TL32711 dosed at 60 mg/kg exerted significant growth arrest/inhibition in 6 primary tumors (T/C range −0.1 to 0.2) and suboptimal growth inhibition in 2 (T/C ˜0.4). H&E slides of resected pancreatic cancer specimens for 7 donor patients were available for evaluation, and there was no relationship between histological findings (inflammatory infiltrate, stroma, neutrophil/lymphocyte ratio and necrosis) and in vivo TL32711 efficacy. Dose escalation studies showed a dose-dependent growth inhibitory effect of TL32711 in 2 primary tumors: 30 mg/kg achieved significant growth inhibition in #17624 but not #12872. Significant growth inhibition was achieved in both at >=45 mg/kg. Pharmacokinetic analysis showed that TL32711 efficacy correlated with tumor drug exposure and that tumor concentrations at the effective doses are in the range of what is achievable in tumors in patients at tolerated doses.

### CONCLUSIONS

TL32711 demonstrated significant single agent efficacy in pancreatic cancer that correlated with tumor drug exposure that were at exposure levels achievable in tumors at tolerated doses in clinical studies.

Explanations of mechanisms of action herein are intended to facilitate understanding of the invention but are not meant to be binding or limiting. It is to be understood that the examples and embodiments described herein are for illustrative purposes only and that various modifications or changes in light thereof will be suggested to persons skilled in the art and are to be included within the spirit and purview of this application and the scope of the appended claims. All references cited hereinabove are incorporated herein by reference as though fully set forth.

1. A method of treating a proliferative disorder in a human patient that comprises internally administering to the patient Compound 15 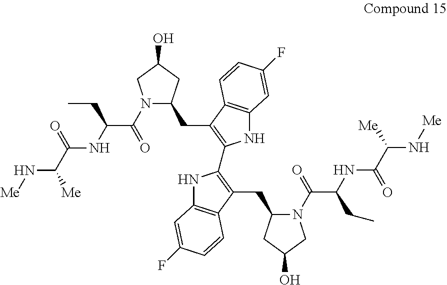 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]
in an amount of 1 to 80 mg/m² of patient body surface area (BSA) by intravenous infusion over a period of 1 to 120 minutes on a weekly or biweekly schedule.

2. The method of claim 1 wherein the amount of Compound 15 administered per dose is 2 to 65 mg/m² and the period of infusion is 1 to 60 minutes.

3. The method of claim 1 wherein the amount of Compound 15 administered per dose is 5 to 65 mg/m² and the period of infusion is 1 to 60 minutes.

4. The method of claim 1 wherein the amount of Compound 15 administered per dose is >30 to 65 mg/m² and the period of infusion is 1 to 60 minutes.

5. The method of claim 1 wherein the amount of Compound 15 administered per dose is 45 to 50 mg/m².

6. The method of claim 1 wherein Compound 15 is administered once, twice, or thrice per week in accordance with a treatment cycle of one, two, three or four weeks on and one week off.

7. The method of claim 6 wherein Compound 15 is administered once per week.

8. The method of claim 6 wherein Compound 15 is administered twice per week.

9. The method of claim 1 wherein Compound 15 is administered once, twice, or thrice per week continuously.

10. The method of claim 9 wherein Compound 15 is administered once per week.

11. The method of claim 9 wherein Compound 15 is administered twice per week.

12. The method of claim 1 wherein the amount of Compound 15 administered per dose is >30 mg/m², and the compound is administered by intravenous infusion during a period of about 30 minutes once per week for three or four weeks on and one week off or continuously.

13. The method of claim 1 wherein the amount of Compound 15 administered per dose is >30 to 65 mg/m², and the compound is administered by intravenous infusion during a period of about 30 minutes once per week, twice weekly, or three times weekly, for three or four weeks on and one week off or continuously.

14. The method of claim 1 wherein the proliferative disorder is a cancer selected from the group consisting of: lung adenocarcinoma, pancreatic cancer, colon cancer, ovarian cancer, breast cancer, mesothelioma, peripheral neuroma, bladder cancer, glioblastoma, melanoma, adrenocortical carcinoma, AIDS-related lymphoma, anal cancer, bladder cancer, meningioma, glioma, astrocytoma, breast cancer, cervical cancer, chronic myeloproliferative disorders (e.g., chronic myelogenous leukemia), chronic lymphocytic leukemia, colon cancer, endocrine cancers, endometrial cancer, ependymoma, esophageal cancer, Ewing's sarcoma, extracranial germ cell tumors, extragonadal germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, intraocular melanoma, islet cell carcinoma, Kaposi sarcoma, laryngeal cancer, leukemia, acute lymphoblastic leukemia (ALL), acute myeloid leukemia (AML), lip cancer, oral cavity cancer, liver cancer, male breast cancer, malignant mesothelioma, medulloblastoma, melanoma, Merkel cell carcinoma, metastatic squamous neck cancer, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, oropharyngeal cancer, bone cancers, including osteosarcoma and malignant fibrous histiocytoma of bone, ovarian epithelial cancer, ovarian germ cell tumors, ovarian low malignant potential tumors, pancreatic cancer, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, skin cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

15. The method of claim 14 wherein the proliferative disorder is a cancer selected from the group consisting of: sarcomas, bladder cancer, ovarian cancer, breast cancer, brain cancer, pancreatic cancer, colon cancer, blood cancer, skin cancer, lung cancer, and bone cancer.

16. The method of claim 14 wherein the cancer is selected from colorectal cancer, renal carinoma, pancreatic carcinoma, prostate carcinoma, melanoma, gliobastoma, acute myeloid leukemia, small cell lung cell carcinoma, non-small cell lung carcinoma, rhabdomyosarcoma, and basal cell carcinoma.

17. The method of claim 14 wherein the cancer is selected from chronic myelogenous leukemia, chronic lymphocytic leukemia, hairy cell leukemia, leukemia, acute lymphoblastic leukemia (ALL), and acute myeloid leukemia (AML).

18. The method of claim 17 wherein the proliferative disorder is AML and Compound 15 is administered at a dose of 15 to 20 mg/m², twice per week.

19. The method of claim 1 that comprises administering Compound 15 in combination with a second cancer therapy selected from radiation, chemotherapy, immunotherapy, photodynamic therapy, and combinations thereof.

20. A pharmaceutical dosage unit suitable for infusion over an infusion period of 1 to 60 minutes comprising Compound 15 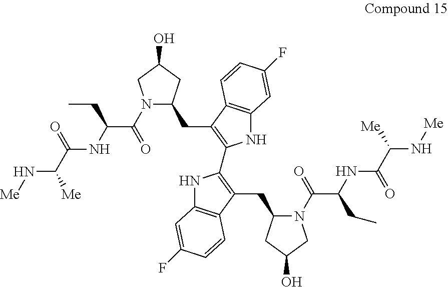 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]
an amount of 1 to 80 mg/m² of patient body surface area (BSA) and a pharmaceutically acceptable carrier or diluent.

21. The pharmaceutical dosage unit of claim 20 suitable for infusion over an infusion period of about 30 minutes comprising Compound 15 and one or more pharmaceutically acceptable excipients in an aqueous solvent for the treatment of a cancer or an autoimmune disorder.

22. Compound 15 for use in the manufacture of a pharmaceutical dosage unit of claim 20.

23. Compound 15 for use in the method of any of claim 1.
